(19) United States
(12) Patent Application Publication
(10) Pub. No.: US 2026/0232306 A1
(43) Pub. Date: Aug. 13, 2026

(54) CONTROL SYSTEM FOR SURGICAL INSTRUMENT

(71) Applicant: Point Robotics Medtech Inc., Hsinchu County (TW)

(72) Inventors: Keng-Hua Yeh, Hsinchu County (TW); TING-YUN FANG, Hsinchu County (TW)

(21) Appl. No.: 19/260,628

(22) Filed: Jul. 7, 2025

(30) Foreign Application Priority Data
Feb. 12, 2025 (TW) 114105106

(51) Int. Cl. A61B 17/02; A61B 90/00; A61B 17/00

(52) U.S. Cl. CPC A61B 17/0218; A61B 90/03; A61B 2017/00075; A61B 2017/00398; A61B 2090/031; A61B 2090/064

(57) ABSTRACT

A control system for a surgical instrument is used to pull a nerve of a patient. The control system includes a force sensor, a conversion element, a force controller, a position controller, a speed controller and a transmission mechanism for driving the surgical instrument. In a collaborative mode of the control system, the force sensor detects the force applied to the surgical instrument, and the conversion element having a dead zone processes an output signal of the force sensor to generate a speed control command for controlling the transmission mechanism. In a constant force mode of the control system, the control system maintains a constant force set by the physician. The control system drives and limits displacement of the surgical instrument, to prevent the nerve from being damaged.

### CROSS-REFERENCE TO RELATED PATENT APPLICATION

This application claims the benefit of priorities to Taiwan Patent Application No.114105106, filed on February 12, 2025. The entire content of the above identified application is incorporated herein by reference.

### FIELD OF THE DISCLOSURE

The present disclosure relates to a control system for a surgical instrument, and more particularly to a control system for assisting a user to stably operate a nerve hook during a spinal decompression surgery.

### BACKGROUND OF THE DISCLOSURE

The spinal decompression surgery is widely used to treat patients with lumbar degeneration or spinal nerve compression. When a physician performs the spinal decompression surgery, in order to create sufficient space for performing the surgery, a nerve hook is used to pull on the nerve near a surgical site and isolate the nerve from the surgical site, so as to avoid accidental injury to the nerve near the spine.

At present, the physician relies entirely on experience to manually operate the nerve hook. However, when surgical time is too long, even an experienced physician cannot maintain at a high level of concentration, so that it can become difficult for the physician to operate the nerve hook with an appropriate force. If the nerve hook pulls the nerve with a too large force or the nerve is pulled by the nerve hook for too long, the nerve hook can injure the nerve near the spine, causing the patient to experience postoperative pain or limb weakness.

### SUMMARY OF THE DISCLOSURE

In response to the above-referenced technical inadequacy, the present disclosure provides a control system of a nerve hook for a spinal decompression surgery.

In order to solve the above-mentioned problem, one of the technical aspects adopted by the present disclosure is to provide a control system for a surgical instrument. The control system includes a force sensor, a conversion element, an encoder, a processing circuit, a speed controller, a force controller, a position controller and a transmission mechanism for driving the surgical instrument. The force sensor is connected between the surgical instrument and the transmission mechanism, and is configured to measure a force condition of the surgical instrument to generate a force sensing signal. The conversion element is configured to convert the force sensing signal into a speed command signal, and the conversion element includes a dead zone. When the force sensing signal is within the dead zone, a speed corresponding to the speed command signal is zero. The encoder is connected to the transmission mechanism and is configured to measure a rotation angle of the transmission mechanism to generate an encoder measurement signal. The processing circuit is configured to generate a position sensing signal and a speed sensing signal of the transmission mechanism according to the encoder measurement signal. The speed controller is configured to generate a speed error signal according to a difference between the speed command signal and the speed sensing signal. The speed error signal is calculated by the speed controller to generate a torque command signal.

In addition, the control system for the surgical instrument includes a processing circuit, a force controller, a position controller and a conversion element. The processing circuit is also configured to generate a force command signal set by a user. The force controller is configured to generate a force error signal according to a difference between the force command signal and the force sensing signal. The force error signal is calculated by the force controller to generate a position command signal. The position controller is configured to generate a position error signal according to a difference between the position command signal and the position sensing signal. The position error signal is calculated by the position controller to generate a speed command signal. The speed command signal is generated by the conversion element or the position controller and then calculated by the speed controller to generate a torque command signal to drive the transmission mechanism.

The first beneficial effect of the control system is that the control system can provide a collaborative mode. In the collaborative mode, when the physician manually operates a nerve hook to pull a nerve, the force sensor measures the physician's force applied to the nerve hook, and cooperates with the speed controller to drive the nerve hook to pull the nerve to a predetermined position at a stable speed. By the dead zone of the speed controller, an erroneous or agitated speed command signal due to noise in the force sensing signal is avoided. When the physician releases the nerve hook, the nerve hook can stay at the place and is not displaced by a reaction force of the nerve.

The second beneficial effect of the control system is that the control system further provides a constant force mode. In the constant force mode, after the physician releases the nerve hook, the nerve hook continues to automatically pull on the nerve with a constant force. The value, time and the interval of the constant force can be set by the physician to best protect the nerve.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a functional block diagram of a control system for a surgical instrument in a collaborative mode according to a first embodiment of the present disclosure;

FIG. 2 is a schematic diagram of a surgical instrument module of FIG. 1 according to one embodiment of the present disclosure;

FIG. 3 is a diagram showing a relationship between a force sensing signal and a speed command signal according to the first embodiment of the present disclosure;

FIG. 4 is a functional block diagram of a speed controller of FIG. 1;

FIG. 5 is a functional block diagram of the control system for the surgical instrument in a constant force mode according to a second embodiment of the present disclosure;

FIG. 6 is a functional block diagram of a force controller of FIG. 5; and

FIG. 7 is a functional block diagram of a position controller of FIG. 5.

### DETAILED DESCRIPTION OF THE EXEMPLARY EMBODIMENTS

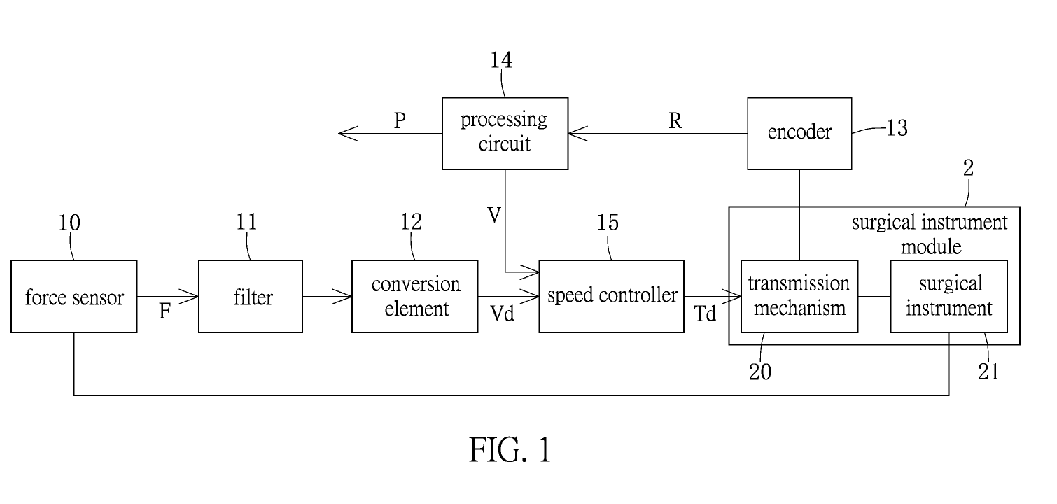

FIG. 1 is a functional block diagram of a control system for a surgical instrument in a collaborative mode according to a first embodiment of the present disclosure. Referring to FIG. 1, the control system includes a force sensor 10, a filter 11, a conversion element 12, an encoder 13, a processing circuit 14 and a speed controller 15. A surgical instrument module 2 includes a transmission mechanism 20 and a surgical instrument 21 connected to the transmission mechanism 20. The surgical instrument 21 is a nerve hook for pulling a nerve. The force sensor 10, the filter 11, the conversion element 12, the encoder 13, the processing circuit 14, the speed controller 15 and the transmission mechanism 20 are electrically connected together in a direct or indirect manner. When a physician manually adjusts the nerve hook (surgical instrument 21) to pull the nerve, the force sensor 10 senses the physician’s force applied to the nerve hook and cooperates with the speed controller 15. The speed controller 15 drives the nerve hook to pull the nerve to a predetermined position at a stable speed.

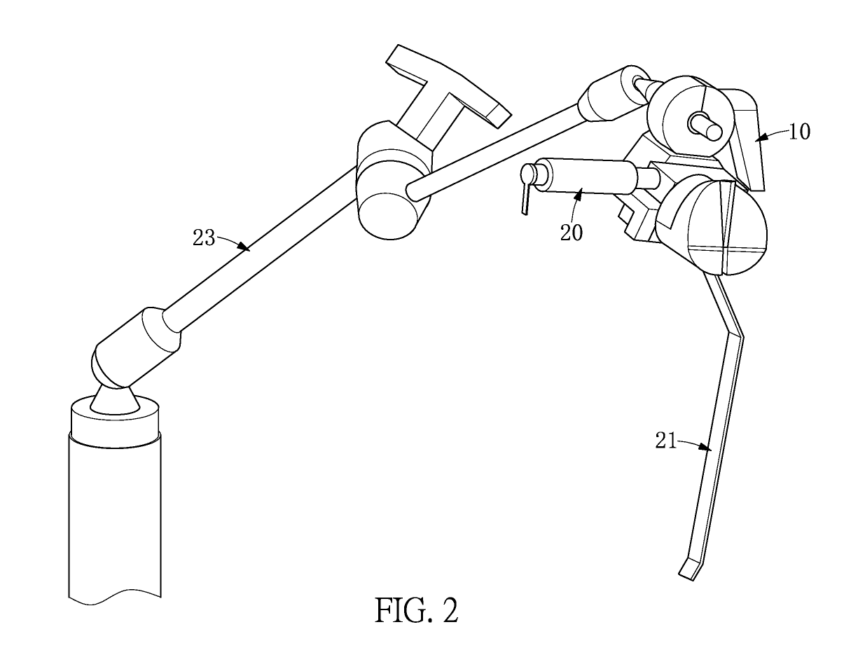

FIG. 2 is a schematic diagram of a surgical instrument module of FIG. 1 according to one embodiment of the present disclosure. Referring to FIG. 2, the transmission mechanism 20 is a DC motor drive device with single degree-of-freedom, and its base is connected to a locking link 23. A movable arm of the transmission mechanism 20 is connected to the surgical instrument 21 via the force sensor 10. The speed controller 15 is electrically connected to the transmission mechanism 20. When the speed controller 15 controls a DC motor of the transmission mechanism 20 to rotate, rotation of the motor drives the movable arm of the transmission mechanism 20 to move, thereby causing the surgical instrument 21 to move to a desired position and/or orientation. The DC motor drive device combining with the locking link 23 shown in FIG. 2 is just one of many ways to drive the surgical instrument 21. The present disclosure can also adopt other driving methods, such as a multi-axis robot arm.

The force sensor 10 is connected between the movable arm of the transmission mechanism 20 and the surgical instrument 21, and is configured to measure a force applied to the surgical instrument 21 to generate a force sensing signal F. The force sensing signal F corresponds to the force applied to the surgical instrument 21. For example, the force sensor 10 may correspond to a six-axis force gauge. The force and torque signals provided by the six-axis force gauge may be used to calculate a magnitude and a direction of the force applied to the surgical instrument 21.

An input end of the filter 11 is connected to an output end of the force sensor 10, and the filter 11 is configured to filter out ripples in the force sensing signal F.

The conversion element 12 may be implemented by, for example, a single chip, and the conversion element 12 is connected to the filter 11. The conversion element 12 is configured to convert the force sensing signal F into a speed command signal Vd, and the conversion element 12 includes a dead zone. The dead zone includes an upper limit and a lower limit. When the force corresponding to the force sensing signal F is within the dead zone, a speed corresponding to the speed command signal Vd converted by the conversion element 12 is zero. By the design of the dead zone, it can avoid generating an erroneous speed command signal Vd due to noise in the force sensing signal F when no force pulls the surgical instrument 21. When a physician releases the nerve hook, the reaction force of the nerve falls within the dead zone, so that the nerve hook can stay at the place and is not displaced by the reaction force of the nerve.

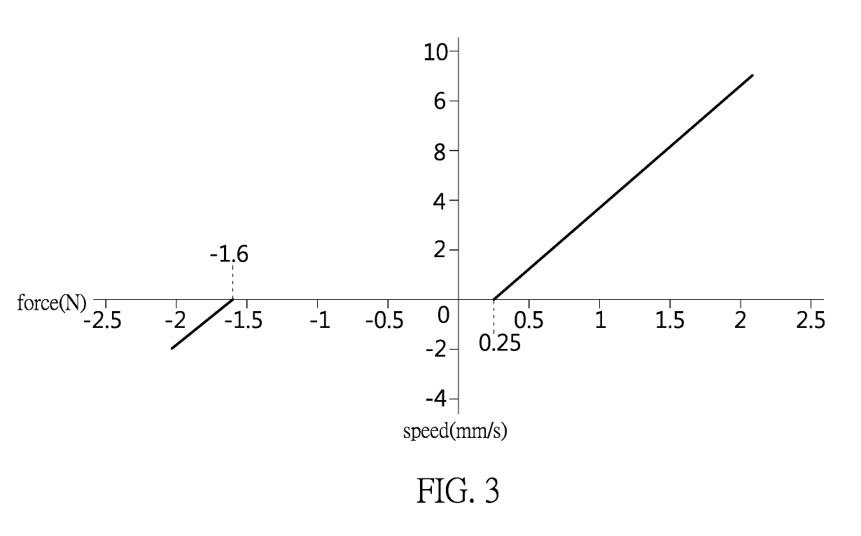

FIG. 3 is a diagram showing a relationship between a force sensing signal and a speed command signal according to the first embodiment of the present disclosure. Referring to FIG. 3, a range of the dead zone of the conversion element 12 is defined as −1.6 newtons (N) to 0.25 newtons (N). When the force corresponding to the force sensing signal F is within the dead zone of the conversion element 12, the speed corresponding to the speed command signal Vd is zero. When the force corresponding to the force sensing signal F is greater than 0.25 newtons, a proportion of “the speed corresponding to the speed command signal Vd” to “the interval above 0.25 newtons of the force sensing signal F” is approximately 5.33:1. When the force corresponding to the force sensing signal F is less than -1.6 N, the proportion of “the speed corresponding to the speed command signal Vd” to “the interval below -1.6 newtons of the force sensing signal F” is approximately 5.33:1. The surgical instrument 21 can be pulled to move to a desired position only when the force corresponding to the force sensing signal F is greater than 0.25 N or less than -1.6 N. In fact, the range of the dead zone and the proportion of the speed to the force outside the dead zone need to be designed according to the dynamic characteristics of the hardware, which are different for different systems.

Referring to FIG. 1, the encoder 13 is connected to the transmission mechanism 20 and is configured to measure a rotation angle of the transmission mechanism 20 to generate an encoder measurement signal R. An input end of the processing circuit 14 is connected to an output end of the encoder 13.

The processing circuit 14 may be, for example, an integrated circuit of a programmable logic controller circuit, a micro-processor circuit, or a micro-control circuit, a central processing unit, etc. The processing circuit 14 is configured to calculate a position sensing signal P of the surgical instrument 21 and a speed sensing signal V of the surgical instrument 21 according to the encoder measurement signal R.

The encoder 13 is connected to the motor of the transmission mechanism 20 to measure a rotation angle of the motor and generate the encoder measurement signal R, wherein the encoder measurement signal R includes rotation angle information of the motor. The processing circuit 14 calculates the position sensing signal P and the speed sensing signal V of the surgical instrument 21 according to the rotation angle information of the motor, where the position sensing signal P includes position information of the surgical instrument 21, and the speed sensing signal V includes speed information of the surgical instrument 21.

An input end of the speed controller 15 is connected to an output end of the conversion element 12 and an output end of the processing circuit 14. The speed controller 15 first calculates a difference between the speed command signal Vd generated by the conversion element 12 and the speed sensing signal V generated by the processing circuit 14, to generate a speed error signal Ve, and then the speed controller 15 generates a torque command signal Td based on the speed error signal Ve.

The speed controller 15 outputs a torque command signal Td to the transmission mechanism 20 so as to control a rotation speed of the motor of the transmission mechanism 20. When the rotation speed of the motor is getting faster, a rotation speed of a lead screw of the transmission mechanism 20 is getting faster, a moving speed of the movable arm of the transmission mechanism 20 is also getting faster, and thus a moving speed of the surgical instrument 21 becomes faster. Due to the dead zone of the conversion element 12, when a physician holds the nerve hook (surgical instrument 21) to pull the nerve, there is a small resistance that slightly resists the physician's force, thereby increasing the stability of pulling the nerve. In addition, when the nerve is pulled to an appropriate position by the nerve hook, the physician can release the nerve hook directly, and the nerve hook can also stay at a released position, thus keeping traction of the nerve as the physician desires.

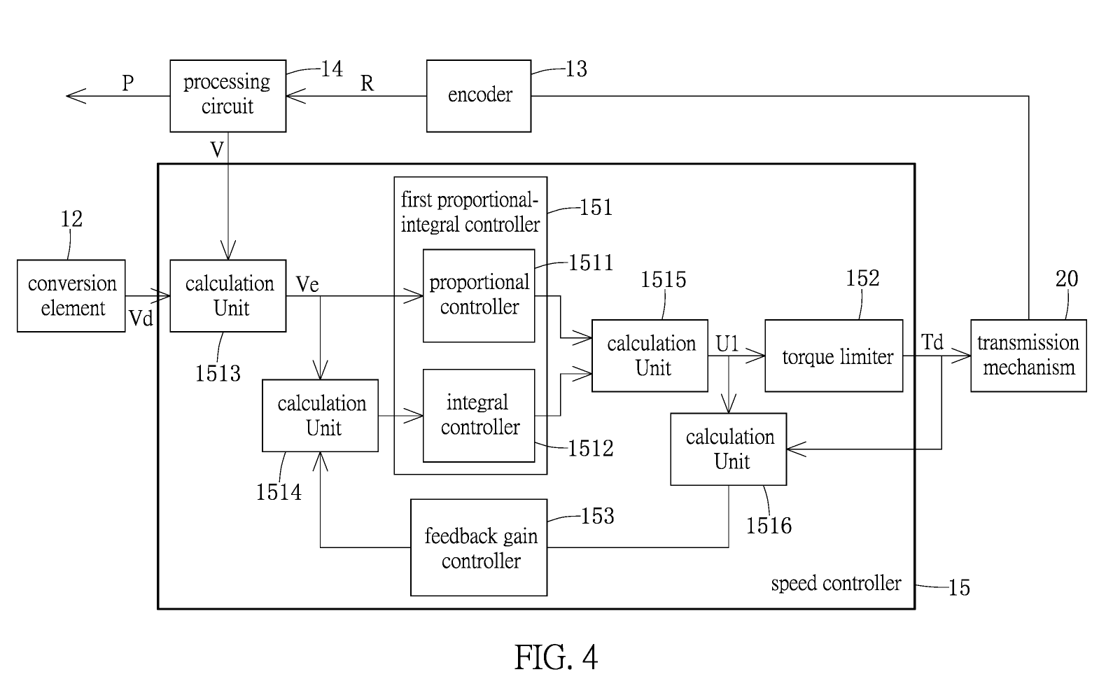

FIG. 4 is a functional block diagram of a speed controller of FIG. 1. Referring to FIG. 4, the speed controller 15 includes a first proportional-integral controller 151, a torque limiter 152, and a feedback gain controller 153. The first proportional-integral controller 151, the torque limiter 152, and the feedback gain controller 153 can be, for example, implemented by a single chip. The first proportional-integral controller 151 includes a proportional controller 1511, an integral controller 1512, and a plurality of calculation units 1513, 1514, 1515, and 1516.

The calculation unit 1513 is connected to the conversion element 12 and the processing circuit 14. The calculation unit 1513 is configured to calculate the difference between the speed command signal Vd and the speed sensing signal V to generate the speed error signal Ve. The calculation unit 1513 is connected to the proportional controller 1511 and the calculation unit 1514. The proportional controller 1511 is configured to multiply the speed error signal Ve by a proportional gain to generate a proportional control signal of the proportional controller 1511. The calculation unit 1514 is connected to the calculation unit 1513 and an output end of the feedback gain controller 153. The calculation unit 1514 is configured to calculate a difference between the speed error signal Ve and an output signal of the feedback gain controller 153 to generate an output signal of the calculation unit 1514. The integral controller 1512 is connected to the calculation unit 1514. The integral controller 1512 is configured to first integrate the output signal of the calculation unit 1514, and then multiply an integration result of the output signal of the calculation unit 1514 by an integral gain to generate an integral control signal of the integral controller 1512. The calculation unit 1515 is connected to the proportional controller 1511 and the integral controller 1512. The calculation unit 1515 is configured to calculate a sum of the proportional control signal of the proportional controller 1511 and the integral control signal of the integral controller 1512 to generate a total control signal U1.

The purpose of the proportional controller 1511 is to provide a fast dynamic response, and the purpose of the integral controller 1512 is to eliminate steady-state errors. In fact, the proportional gain and integral gain need to be designed according to the dynamic characteristics of the hardware, which are different for different systems.

The torque limiter 152 is connected to the calculation unit 1515. The torque limiter 152 defines an upper torque limit and a lower torque limit. When a value corresponding to the total control signal U1 is not greater than the upper torque limit or is not lower than the lower torque limit, a torque corresponding to the torque command signal Td outputted by the torque limiter 152 is equal to the value corresponding to the total control signal U1 outputted by the calculation unit 1515. When the value corresponding to the total control signal U1 is greater than the torque upper limit, the torque corresponding to the torque command signal Td outputted by the torque limiter 152 is equal to the torque upper limit. When the value corresponding to the total control signal U1 is lower than the torque lower limit, the torque corresponding to the torque command signal Td outputted by the torque limiter 152 is equal to the torque lower limit.

The calculation unit 1516 is connected to the calculation unit 1515, the torque limiter 152 and the feedback gain controller 153. The calculation unit 1516 is configured to calculate a difference between the total control signal U1 of the calculation unit 1515 and the torque command signal Td of the torque limiter 152 to generate an output signal of the calculation unit 1516. The feedback gain controller 153 is configured to multiply the output signal of the calculation unit 1516 by a feedback gain to generate an output signal of the feedback gain controller 153. Specifically, the torque limiter 152, the calculation unit 1516 and the feedback gain controller 153 constitute an anti-windup compensator. The main purpose of the anti-windup compensator is to limit the errors accumulated by the integral controller 1512. When the total control signal U1 exceeds a utilizable range, the feedback gain controller 153 can prevent the integral controller 1512 from excessively integrating the error and maintain the stability of an integral part of the total control signal U1. When the total control signal U1 returns to the utilizable range, the response speed of the control system can also be increased.

In summary, during the process of pulling the nerve in the collaborative mode, the physician’s force applied to the nerve hook measured by the force sensor 10 is filtered by the filter 11 and then converted into a target speed of the nerve hook by the conversion element 12. Finally, the speed controller 15 adjusts the moving speed of the nerve hook during the process of pulling the nerve so that it stably reaches the target speed.

When the speed controller 15 drives the nerve hook at a stable speed to pull the nerve to a predetermined position, the physician can release the nerve hook. After the nerve hook is released, the system enters a constant force mode and can drive the nerve hook to automatically pull on the nerve with a constant force.

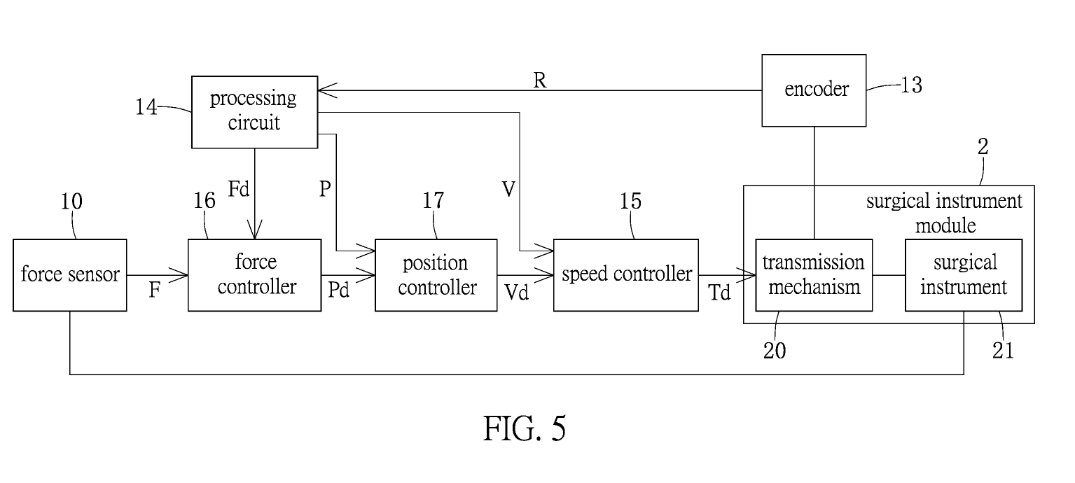

FIG. 5 is a functional block diagram of the control system for the surgical instrument in a constant force mode according to a second embodiment of the present disclosure. Referring to FIG. 5, the difference between FIG. 5 and FIG. 1 is that the control system of FIG. 5 further includes a force controller 16 and a position controller 17, and the speed controller 15 of FIG. 5 is the same as the speed controller 15 of FIG. 4.

In the constant force mode, the processing circuit 14 calculates the position sensing signal P and the speed sensing signal V according to the encoder measurement signal R. The processing circuit 14 also generates a force command signal Fd set by a user, and a force corresponding to the force command signal Fd is a constant value. The force controller 16 first calculates a difference between the force sensing signal F and the force command signal Fd to generate a force error signal, and then generates a position command signal Pd according to the force error signal. Specifically, the force sensing signal F corresponds to the force applied on the nerve hook by the nerve and measured by the force sensor 10.

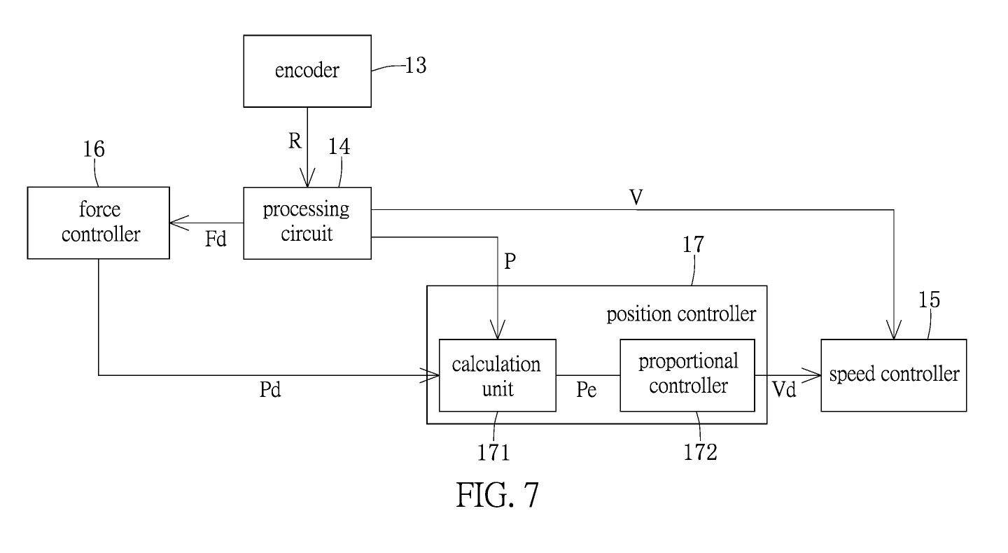

The position controller 17 first calculates a difference between the position command signal Pd generated by the force controller 16 and the position sensing signal P generated by the processing circuit 14 to generate a position error signal Pe (shown in FIG. 7), and then calculates the speed command signal Vd according to the position error signal Pe.

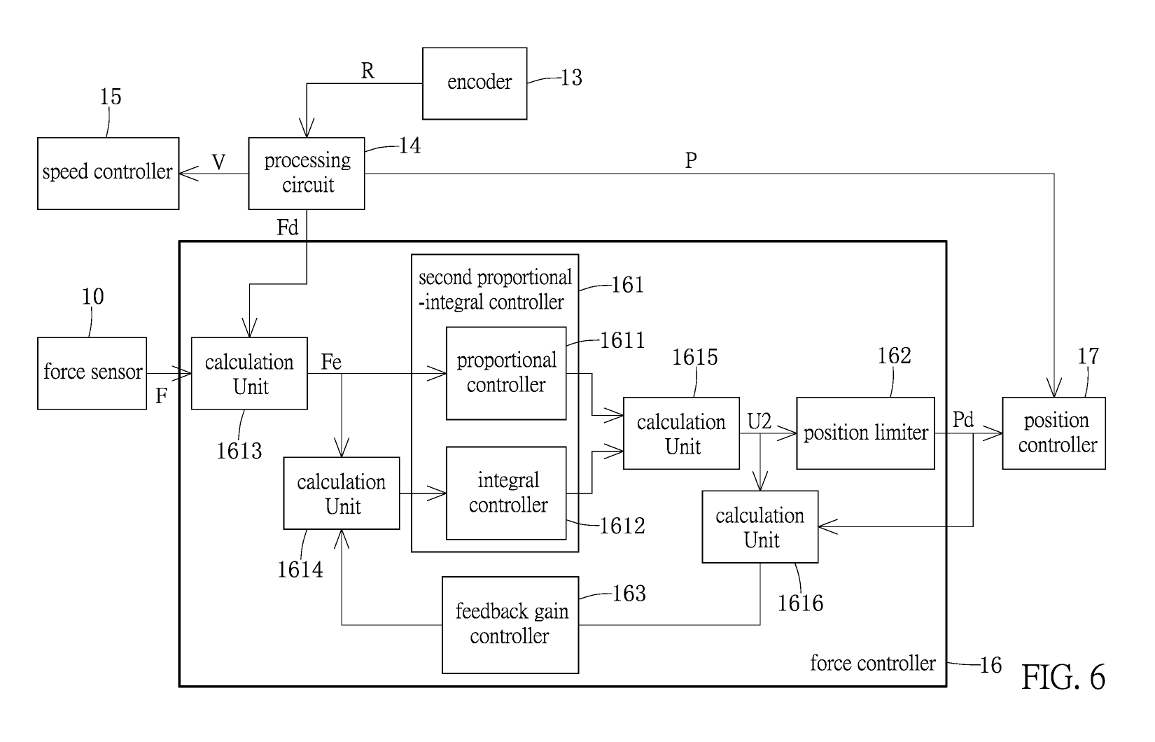

FIG. 6 is a functional block diagram of a force controller of FIG. 5. Referring to FIG. 6, the force controller 16 includes a second proportional-integral controller 161, a position limiter 162, and a feedback gain controller 163. The second proportional-integral controller 161, the position limiter 162, and the feedback gain controller 163 can be, for example, implemented by a single chip. The second proportional-integral controller 161 includes a proportional controller 1611, an integral controller 1612, and a plurality of calculation units 1613, 1614, 1615, and 1616. The calculation unit 1613 is connected to the force sensor 10 and the processing circuit 14. The calculation unit 1613 is configured to calculate a difference between the force sensing signal F and the force command signal Fd to generate a force error signal Fe. The proportional controller 1611 is connected to the calculation unit 1613. The proportional controller 1611 is configured to multiply the force error signal Fe by a proportional gain to generate a proportional control signal of the proportional controller 1611. The calculation unit 1614 is connected to the calculation unit 1613 and an output end of the feedback gain controller 163. The calculation unit 1614 is configured to calculate a difference between the force error signal Fe and an output signal of the feedback gain controller 163 to generate an output signal of the calculation unit 1614. The integral controller 1612 is connected to the calculation unit 1614. The integral controller 1612 first integrates the output signal of the calculation unit 1614, and then multiplies an integration result of the output signal of the calculation unit 1614 by an integral gain to generate an integral control signal of the integral controller 1612. The calculation unit 1615 is connected to the proportional controller 1611 and the integral controller 1612. The calculation unit 1615 is configured to calculate a sum of the proportional control signal of the proportional controller 1611 and the integral control signal of the integral controller 1612 to generate a total control signal U2. In fact, the proportional gain and the integral gain need to be designed according to the dynamic characteristics of the hardware, which are different for different systems.

The position limiter 162 is connected to the calculation unit 1615. The position limiter 162 defines an upper position limit and a lower position limit. When a value corresponding to the total control signal U2 is not greater than the upper position limit or is not lower than the lower position limit, a position corresponding to the position command signal Pd outputted by the position limiter 162 is equal to the value corresponding to the total control signal U2 outputted by the calculation unit 1615. When the value corresponding to the total control signal U2 is greater than the upper position limit, the position corresponding to the position command signal Pd outputted by the position limiter 162 is equal to the upper position limit. When the value corresponding to the total control signal U2 is lower than the position lower limit, the position corresponding to the position command signal Pd outputted by the position limiter 162 is equal to the lower position limit.

Specifically, the position limiter 162, the calculation unit 1616 and the feedback gain controller 163 together constitute an anti-windup compensator. The anti-windup compensator is used for two purposes. The first purpose is to limit the error accumulated by the integral controller 1612. When the total control signal U2 exceeds a utilizable range, the feedback gain controller 163 can prevent the integral controller 1612 from excessively integrating the error and maintain the stability of an integral part of the total control signal U2. When the total control signal U2 returns to the utilizable range, the response speed of the control system can also be increased. The second purpose is to limit a maximum displacement of the nerve hook under the control of the constant force, so as to avoid excessive movement of the nerve hook due to erroneous touch, interference or other unexpected accident caused by the user, which may result in harm to the patient. The calculation unit 1616 is connected to the calculation unit 1615, the position limiter 162 and the feedback gain controller 163. The calculation unit 1616 is configured to calculate a difference between the total control signal U2 of the calculation unit 1615 and the position command signal Pd of the position limiter 162 to generate an output signal of the calculation unit 1616. The feedback gain controller 163 is configured to multiply the output signal of the operation unit 1616 by a feedback gain to generate an output signal of the feedback gain controller 163.

FIG. 7 is a functional block diagram of a position controller of FIG. 5. Referring to FIG. 7, the position controller 17 includes a calculation unit 171 and a proportional controller 172. The calculation unit 171 and the proportional controller 172 can be, for example, implemented by a single chip. The calculation unit 171 is connected to the force controller 16 (the output end of the position limiter 162), the output end of the processing circuit 14, and the input end of the proportional controller 172. The calculation unit 171 is configured to calculate a difference between the position command signal Pd of the position limiter 162 and the position sensing signal P of the processing circuit 14 to generate a position error signal Pe. The proportional controller 172 is connected to the output end of the calculation unit 171. The proportional controller 172 is configured to multiply the position error signal Pe by a proportional gain to generate a speed command signal Vd. The proportional controller 172 outputs the speed command signal Vd to the speed controller 15.

### Beneficial Effects of the Embodiments

The first beneficial effect is described as follows. In the collaborative mode of the control system, the control system can assist the physician to pull the nerve to a predetermined position at a stable speed during the traction process. By the dead zone of the speed controller, an erroneous speed command signal due to noise in the force sensing signal is avoided. As the nerve is pulled to the predetermined position and the physician releases the nerve hook, the reaction force of the nerve is still within the dead zone, therefore the nerve hook can stay at the place and is not displaced by the reaction force of the nerve.

The second beneficial effect is that the control system can initiate the constant force mode as the nerve hook is moved to the predetermined position, so as to automatically maintain a constant traction force on the nerve. Even if the nerve hook encounters a sudden collision, the force controller keeps traction force of the nerve hook at a roughly constant value, which can prevent the nerve hook from suddenly pulling the nerve with an excessive force. When the nerve hook is displaced due to a sudden collision, the position controller can limit the displacement of the nerve hook, to prevent the excessive displacement of the nerve hook from damaging the nerve. The value, time and the interval of the constant force can be set by the physician, which allows the physician to pull the nerve with an accurate force and release the nerve hook at an appropriate time, to avoid damaging the nerve.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A control system for a surgical instrument, applied to control a transmission mechanism for driving the surgical instrument, the control system comprising:
a force sensor connected between the surgical instrument and the transmission mechanism and being configured to measure a force condition of the surgical instrument to generate a force sensing signal;
a conversion element configured to convert the force sensing signal into a speed command signal, wherein the conversion element includes a dead zone, and when the force sensing signal is within the dead zone, a speed corresponding to the speed command signal is zero;
an encoder connected to the transmission mechanism and configured to measure a rotation angle of the transmission mechanism to generate an encoder measurement signal;
a processing circuit configured to generate a position sensing signal of the transmission mechanism and a speed sensing signal of the transmission mechanism according to the encoder measurement signal; and
a speed controller configured to calculate a difference between the speed command signal and the speed sensing signal to generate a speed error signal, wherein the speed error signal is calculated by the speed controller to generate a torque command signal.

2. The control system according to claim 1, further comprising a filter, wherein the filter is connected between the force sensor and the conversion element, and the filter filters out ripples in the force sensing signal.

3. The control system according to claim 1, wherein the speed controller includes a first proportional-integral controller, at least one calculation unit, a feedback gain controller and a torque limiter, the first proportional-integral controller is connected to the torque limiter, the torque limiter outputs the torque command signal to the transmission mechanism, the torque limiter is configured to limit upper and lower limits of a torque of the transmission mechanism, an output of the torque limiter is processed by the at least one calculation unit and the feedback gain controller and then input to the first proportional-integral controller.

4. The control system according to claim 1, further comprising a force controller, wherein the processing circuit generates a force command signal, and a force corresponding to the force command signal is a constant value, the force controller is configured to calculate a difference between the force sensing signal and the force command signal to generate a force error signal, and the force error signal is calculated by the force controller to generate a position command signal.

5. The control system according to claim 4, wherein the force controller includes a second proportional-integral controller, at least one calculation unit, a feedback gain controller and a position limiter, the second proportional-integral controller is connected to the position limiter, the position limiter outputs the position command signal, the position limiter is configured to limit upper and lower limits of a position of the transmission mechanism, an output of the position limiter is processed by the at least one calculation unit and the feedback gain controller and then input to the second proportional-integral controller.

6. The control system according to claim 4, further comprising a position controller, wherein the processing circuit is configured to generate the position sensing signal of the transmission mechanism according to the rotation angle of the transmission mechanism, and the position controller is configured to calculate a difference between the position command signal and the position sensing signal to generate a position error signal, and the position error signal is calculated by the position controller to generate the speed command signal.

7. The control system according to claim 6, wherein the position controller includes a proportional controller, an input of the proportional controller is the position error signal, and the proportional controller outputs the speed command signal to the speed controller.
